(19) United States
(12) Patent Application Publication
(10) Pub. No.: US 2026/0166058 A1
(43) Pub. Date: Jun. 18, 2026

(54) Method for Treating or Reducing Localized Fat and Pharmaceutical Composition for Use in Treating or Reducing Localized Fat

(71) Applicant: Rimonim Personal Clinics Ltd., Kiryat Tiv'on (IL)

(72) Inventors: Kfir Zroya, Modi'in-Maccabim-Re'ut (IL); Alon Edelman, Holon (IL); Dror Asraf, Kiryat Tiv'on (IL)

(21) Appl. No.: 19/422,870

(22) Filed: Dec. 17, 2025

Related U.S. Application Data
(60) Provisional application No. 63/734,865, filed on Dec. 17, 2024.

(51) Int. Cl. A61K 31/575; A61K 9/00; A61K 47/36; A61P 3/04

(52) U.S. Cl. CPC A61K 31/575; A61K 9/0019; A61K 47/36; A61P 3/04

(57) ABSTRACT

A method for treating or reducing localized fat, and a pharmaceutical composition for use in treating or reducing localized fat. The method comprises injecting the pharmaceutical composition into the localized fat of a subject in need, wherein the pharmaceutical composition comprises deoxycholic acid and a viscous agent, and wherein deoxycholic acid is present in the pharmaceutical composition in an amount of 1% to 5% w/v.

### CROSS-REFERENCE TO RELATED APPLICATIONS

This application claims priority from U.S. Provisional Patent Application No. 63/734,865, filed 17/12/2024, the disclosure of which is incorporated by reference in its entirety herein.

### TECHNICAL FIELD

The present invention relates to aesthetic medicine. It discloses method and pharmaceutical composition for use in treating or reducing localized fat deposits, such as those in the “double chin” and abdominal areas through lipolytic process.

### BACKGROUND OF THE INVENTION

Aesthetic medicine is a rapidly expanding field in the developed world, with increasing use of various modalities and tools such as hyaluronic acid (HA) and bio-stimulators to enhance skin texture, pigmentation, and tightness. As the market grows, more research is being conducted on innovative injection techniques and products, often with synergistic effects when combined. With advancing research, new approaches are being found to achieve better patient outcomes with fewer side effects, particularly within this less invasive alternative to surgical procedures.

A common need has emerged for effective solutions to target localized fat deposits, such as those in the “double chin” and abdominal areas. Based on extensive research, rheological principles (the science of flow and deformation of matter, both solids and liquids), and the application of various deoxycholic acid products

Currently, there are two primary options for fat reduction:
Invasive methods (e.g., liposuction)
Non-invasive methods (e.g., injectables)

In the non-invasive category, two main chemicals are used: deoxycholic acid and phosphatidylcholine. Research and product testing indicate that phosphatidylcholine often presents excessive side effects with limited lipolytic (fat-dissolving) benefits, making its risks outweigh its advantages. Side effects such as itching and significant swelling negatively impact the patient experience, reducing demand for such treatments. This is likely why most leading products contain deoxycholic acid without phosphatidylcholine.

Deoxycholic acid functions as a lipolytic agent by disrupting the cell membranes of fat cells (adipocytes), leading to irreversible tissue damage. After cell death, an inflammatory response removes cell debris, releases intracellular fat, and recruits fibroblasts for collagen formation.

There are two main types of deoxycholic acid products on the market, both containing 1% deoxycholic acid, which often fails to deliver the optimal results clients seek, potentially leading to frustration. The primary difference between these products is their rheological properties. The first type is a liquid with no viscosity, while the second type combines deoxycholic acid with hyaluronic acid to increase viscosity. The increased viscosity allows for better control during injections, as more viscous products resist spreading away from injection points. This feature helps prevent uneven fat dissolution, where some areas dissolve while others remain unaffected.

The more viscous product type is safer and has fewer side effects because it does not contain phosphatidylcholine, and it provides the injector with greater control. However, a drawback of these products is that hyaluronic acid absorbs water, causing swelling through a process known as “hydration swelling.” The more inflamed the tissue, the more water is absorbed, which exacerbates swelling. This limitation restricts these formulas to lower deoxycholic acid concentrations, ultimately compromising optimal results.

Prolotherapy and mesotherapy are treatments aimed at improving tissue quality. Prolotherapy is used primarily for injuries to tendons and ligaments, while mesotherapy is used to enhance skin quality, mainly targeting the dermis layer but also the epidermis.

Research on prolotherapy and mesotherapy shows that these treatments stimulate fibrocytes, the cells responsible for tissue repair and rejuvenation, to gather at the injection site where the active substance is located. In prolotherapy, fibrocytes are directed toward repairing injured tissue, while in mesotherapy, they rejuvenate healthy tissue. Both treatments promote collagen production.

Though prolotherapy and mesotherapy have different goals and target distinct anatomical and histological locations, there is no documented use of prolotherapeutic substances, such as dextran, for mesotherapy purposes.

### SUMMARY OF THE INVENTION

The present invention is a method for treating or reducing localized fat and pharmaceutical composition for use in treating or reducing localized fat.

According to the teachings of an embodiment of the present invention, there is provided a method for treating or reducing localized fat and pharmaceutical composition for use in treating or reducing localized fat, wherein the pharmaceutical composition comprises deoxycholic acid and a viscous agent, and wherein deoxycholic acid in the pharmaceutical composition is present in an amount from 1% to 5% w/v.

In certain embodiment of the current invention, the viscous agent is polysaccharide.

In certain embodiment of the current invention, the polysaccharide is dextran polymer.

In certain embodiment of the current invention, the dextran polymer is present in an amount from 2 to 8% (w/v).

Unless otherwise defined herein, all technical and/or scientific terms used herein have the same meaning as commonly understood by one of ordinary skill in the art to which the invention pertains. Although methods and materials similar or equivalent to those described herein may be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the present invention are herein described, by way of example only, with reference to the accompanying drawings. With specific reference to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

Attention is now directed to the drawings, where like reference numerals or characters indicate corresponding or like components. In the drawings:

FIG. 1 shows the effect obtained after five injections of 3 mL each of a 4% w/w deoxycholic acid pharmaceutical composition, as described in Embodiment 3. The Top image shows the condition prior to injection, and the bottom image shows the condition one month after injection.

FIG. 2 shows the effect obtained after a single injection of 1 mL of a 2% w/w deoxycholic acid pharmaceutical composition, as described in Embodiment 3. The left image shows the condition prior to injection, and the right image shows the condition one month after injection.

FIG. 3 shows the effect obtained after a single injection of 1 mL of a 2% w/w deoxycholic acid pharmaceutical composition, as described in Embodiment 3. The left image shows the condition prior to injection, and the right image shows the condition one month after injection.

FIG. 4 shows the effect obtained after a single injection of 1 mL of a 2% w/w deoxycholic acid pharmaceutical composition, as described in Embodiment 3. The left image shows the condition prior to injection, and the right image shows the condition one month after injection.

### DESCRIPTION OF THE PREFERRED EMBODIMENTS

The present invention is a method for treating or reducing localized fat and pharmaceutical composition for use in treating or reducing localized fat.

The principles and operation of method for treating or reducing localized fat according to present invention may be better understood with reference to the drawings accompanying the description.

Before explaining at least one embodiment of the invention in detail, it is to be understood that the invention is not necessarily limited in its application to the details of construction and the arrangement of the components and/or methods set forth in the following description and/or illustrated in the drawings and/or the examples. The invention is capable of other embodiments or of being practiced or carried out in various ways.

By way of introduction, Dextran polymer does not readily adsorb water molecules resulting from the inflammatory process, which allows for the use of higher deoxycholic acid concentrations. Additionally, dextran slows the dissolution of fat tissues by shielding the fat tissues from deoxycholic acid molecules. Over time, as dextran polymer gradually breaks down, the polymer exposes fat cells to unreacted deoxycholic acid. The slower release process reduces side effects from rapid changes, giving the body time to adapt. For example, rapid volume loss in the body can lead to stretch marks and loose skin, similar to the marks left after childbirth. This process is somewhat like the function of dietary fiber, which lowers the glycemic index by slowing glucose interaction with gastrointestinal cells, thus delaying absorption into the bloodstream.

Loss of subcutaneous fat, which supports skin tone and tightness, is an unwanted side effect of treating localized fat using a formula based solely on deoxycholic acid. Adding dextran helps reduce this effect by increasing the amount of specific collagen responsible for maintaining skin tone and tightness.

Twenty-eight types of human collagen have been identified, described, and categorized into groups based on the structures they form. Histologically, collagen types 17, 4, and 7 are arranged vertically from the epidermis to the dermis in the layer known as the DEJ (Dermal-Epidermal Junction), while collagen types 1 and 3 are arranged horizontally. The vertically arranged collagens contribute to the tightness and texture of the skin. Studies have shown that using dextran reduces procollagen in the medium and dramatically increases the amount of collagen associated with the cell layer (collagen types 17, 4, and 7). This suggests that a lipolytic effect is achieved, while skin tone and tightness improve due to an increase in the specific collagen types responsible for these properties. Additionally, the levels of collagen types 1 and 3 also increase in response to the treatment.

The disclosed method and formula offer the following advantages:
A higher percentage of deoxycholic acid than that found in current available commercial products which leads to improved cosmetic results (more fat dissolved).
Greater viscosity than that found in current available commercial products, allowing for better control during injection.
Prevention of swelling from water absorption, enabling the use of higher deoxycohlic concentrations.
Prevention of side effects like itch or pain

Additionally, the disclosed method involves a slower fat-dissolving process, reducing side effects such as stretch marks and loose skin. This approach enhances skin tone and tightness along with providing a lipolytic effect.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

As used herein, the singular form “a”, “an” and “the” include plural references unless the context clearly dictates otherwise.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Embodiments of the present invention are directed to a method and a pharmaceutical composition for treating or reducing localized fat. The pharmaceutical composition has been developed to minimize side effects while allowing the safe use of higher active ingredient concentrations. This formula is presented here with the purpose of enhancing fat reduction results with increased safety. The method comprises steps of injecting a substance to the localized fat, wherein the substance comprises aqueous solution of deoxycholic acid and viscous agent

### Embodiment 1: Pharmaceutical Composition

In one embodiment, the present invention provides a pharmaceutical composition for treating or reducing localized fat. The composition comprises deoxycholic acid and a viscous agent. In particular, deoxycholic acid is present in the composition in an amount ranging from 1% to 5% w/v. The viscous agent may include polysaccharide, and in some embodiments, polysaccharide is a dextran polymer. In certain embodiments of the present invention, the dextran has a molecular weight in the range of 80-140 kDa, with sub-ranges within this range also applicable, for example 104.17-119.04 kDa. The dextran polymer may be present in the composition in an amount ranging from about 2% to about 8% w/v.

In particular embodiments, when the dextran polymer is present at 5% w/v, the pharmaceutical composition has a viscosity in the range of 6-9 cPoise and an osmolarity in the range of 400-600 mOsmol.

### Embodiment 2: Method of Use

The pharmaceutical composition may be administered to a subject in need thereof by direct injection into localized fat deposits. According to the teachings of an embodiment of the present invention, there is provided a method for treating or reducing localized fat, comprising injecting a pharmaceutical composition into the localized fat, wherein the pharmaceutical composition comprises deoxycholic acid and a viscous agent, and wherein deoxycholic acid in the pharmaceutical composition is present in an amount from 1% to 5% w/v. The viscous agent may include polysaccharide, and in some embodiments, polysaccharide is a dextran polymer. In certain embodiments of the present invention, the dextran has a molecular weight in the range of 80-140 kDa, with sub-ranges within this range also applicable, for example 104.17-119.04 kDa. The dextran polymer may be present in the composition in an amount ranging from about 2% to about 8% w/v.

In particular embodiments, when the dextran polymer is present at 5% w/v, the pharmaceutical composition has a viscosity in the range of 6-9 cPoise and an osmolarity in the range of 400-600 mOsmol.

### Embodiment 3: Preparation of 2% w/w and 4% w/w Deoxycholic Acid Pharmaceutical Composition

In one embodiment, the present invention provides a method for preparing a pharmaceutical composition for treating or reducing localized fat, comprising the steps of:
Dissolving 1 g or 2 g of deoxycholic acid in 20-25 mL of Water for Injection (WFI), and titrating the solution with 1 N NaOH until complete dissolution, resulting in a pH of about 8.0 to 8.3;
Adding 0.45 g of benzyl alcohol to the deoxycholic acid solution;
Preparing a dextran solution by suspending 2.5 g or 3 g of dextran in 15 mL of WFI containing 52.6 mg of disodium dihydrogen phosphate and 3.5 mg of monosodium dihydrogen phosphate, heating the solution to 30-40° C. until complete dissolution, and adding 10 mg of disodium EDTA;
Cooling the dextran solution to room temperature and combining it with the deoxycholic acid solution, mixing until a homogeneous composition is obtained;
Adjusting the pH of the final composition to about 7.5 to 8.3 and the osmolality to about 270-330 mOsmol by adding NaCl;
Bringing the total weight of the composition to 50 g; and
Sterilizing the composition by filtration through a 0.22 μm Nylon filter.

### Embodiment 4: Pharmaceutical Composition for Use in Treating or Reducing Localized Fat.

In certain embodiments, the pharmaceutical compositions according to the present invention (presented in Embodiments 1 and 3) are used in treating or reducing localized fat of subject in need.

Referring Now to the Drawings,

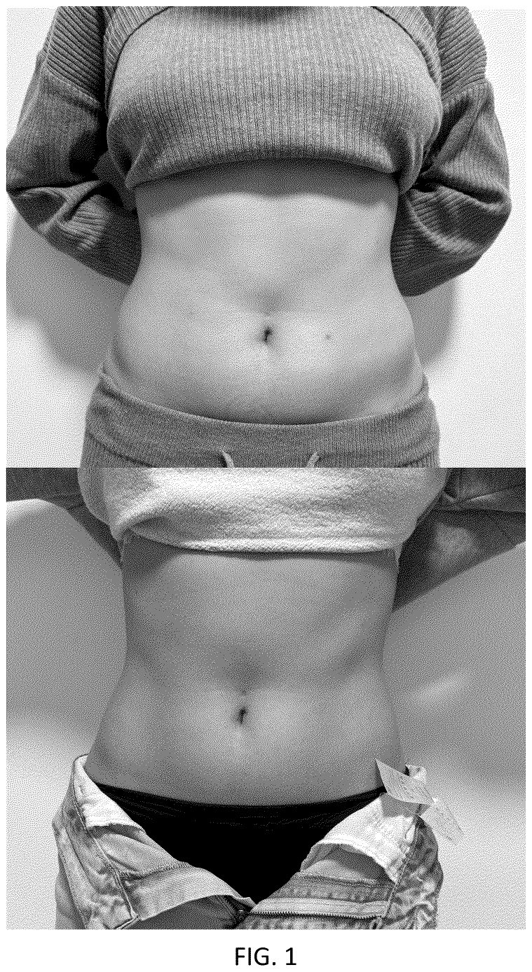

FIG. 1 shows the effect obtained after five injections of 3 mL each of a 4% w/w deoxycholic acid pharmaceutical composition, as described in Embodiment 3. The Top image shows the condition prior to injection, and the bottom image shows the condition one month after injection.

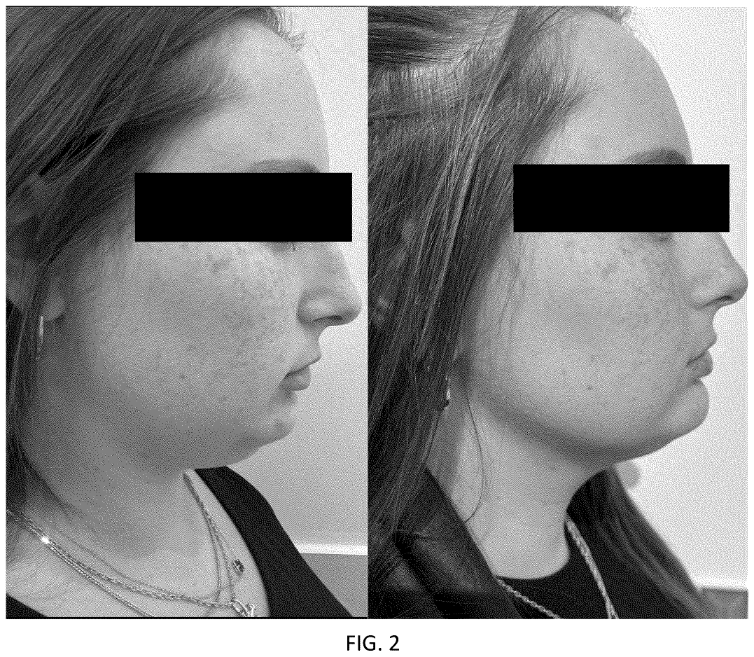

FIG. 2 shows the effect obtained after a single injection of 1 mL of a 2% w/w deoxycholic acid pharmaceutical composition, as described in Embodiment 3. The left image shows the condition prior to injection, and the right image shows the condition one month after injection.

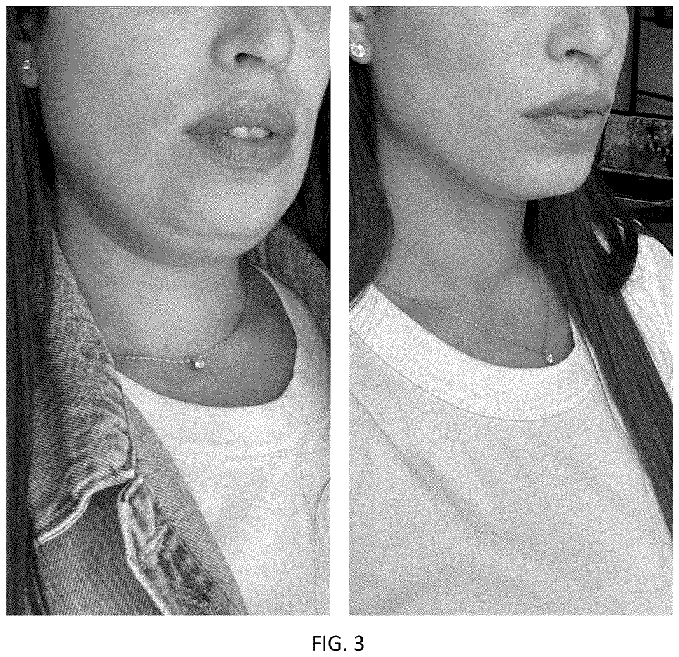

FIG. 3 shows the effect obtained after a single injection of 1 mL of a 2% w/w deoxycholic acid pharmaceutical composition, as described in Embodiment 3. The left image shows the condition prior to injection, and the right image shows the condition one month after injection.

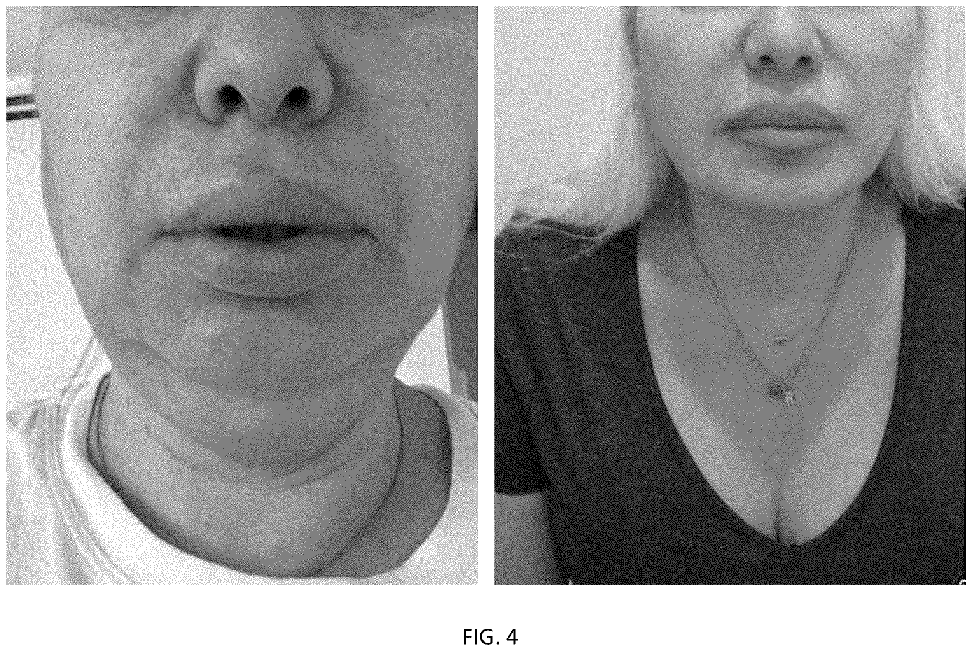

FIG. 4 shows the effect obtained after a single injection of 1 mL of a 2% w/w deoxycholic acid pharmaceutical composition, as described in Embodiment 3. The left image shows the condition prior to injection, and the right image shows the condition one month after injection.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

As used herein, the singular form, “a”, “an” and “the” include plural references unless the context clearly dictates otherwise.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A method for treating or reducing localized fat, comprising injecting a pharmaceutical composition into the localized fat, wherein the pharmaceutical composition comprises deoxycholic acid and a viscous agent, and wherein deoxycholic acid in the pharmaceutical composition is present in an amount from 1% to 5% w/v.

2. The method according to claim 1, wherein the viscous agent is polysaccharide.

3. The method according to claim 2, wherein the polysaccharide is dextran polymer.

4. The method according to claim 3, wherein the dextran polymer is present in an amount from 2 to 8% (w/v).

5. A pharmaceutical composition comprising deoxycholic acid and a viscous agent, wherein deoxycholic acid is present in an amount from 1 to 5% (w/v).

6. The pharmaceutical composition according to claim 5, wherein the viscous agent is polysaccharide.

7. The pharmaceutical composition according to claim 6, wherein the polysaccharide is dextran polymer.

8. The pharmaceutical composition according to claim 7, wherein the dextran polymer is present in an amount from 2 to 8% (w/v).

9. The pharmaceutical composition according to claim 5 for use in treating or reducing localized fat.

10. The pharmaceutical composition according to claim 6 for use in treating or reducing localized fat.

11. The pharmaceutical composition according to claim 7 for use in treating or reducing localized fat.

12. The pharmaceutical composition according to claim 8 for use in treating or reducing localized fat.
